(19) United States
(12) Patent Application Publication
(10) Pub. No.: US 2020/0251178 A1
(43) Pub. Date: Aug. 6, 2020

(54) Method and System for Identifying Clinical Phenotypes in Whole Genome DNA Sequence Data

(71) Applicant: The Board of Trustees of the Leland Stanford Junior University, Stanford, CA (US)

(72) Inventors: Frederick Dewey, Redwood City, CA (US); Euan A. Ashley, Stanford, CA (US); James Priest, Stanford, CA (US); Megan Grove, Stanford, CA (US)

(73) Assignee: The Board of Trustees of the Leland Stanford Junior University, Stanford, CA (US)

(21) Appl. No.: 16/780,249

(22) Filed: Feb. 3, 2020

Related U.S. Application Data
(63) Continuation of application No. 14/645,312, filed on Mar. 11, 2015.
(60) Provisional application No. 61/950,957, filed on Mar. 11, 2014.

(51) Int. Cl. G16B 20/00; G16B 30/00

(52) U.S. Cl. CPC G16B 20/00; G16B 30/00

(57) ABSTRACT

High throughput sequencing has facilitated a precipitous drop in the cost of whole genome human DNA sequencing, prompting predictions of a revolution in medicine via personalization of diagnostic and therapeutic strategies to individual genetics. Disclosed is a comprehensive series of methods for identification of genetic variants and medical genotypes, phasing genetic data and using Mendelian inheritance for quality control, and providing predictive genetic information about risk for rare disease phenotypes and response to pharmacological therapy in single individuals and father-mother-child trios.

### CROSS-REFERENCE TO RELATED APPLICATIONS

This current application is a continuation of U.S. patent application Ser. No. 14/645,312, filed Mar. 11, 2015, entitled “Method and System for Identifying Clinical Phenotypes in Whole Genome DNA Sequence Data” to Dewey et al., which claims priority to U.S. Provisional Application No. 61/950,957 filed Mar. 11, 2014, the disclosures of which are expressly incorporated by reference herein in their entirety.

### GOVERNMENT RIGHTS

This invention was made with Government support under contracts HD000850 and HL094274 awarded by the National Institutes of Health. The Government has certain rights in the invention.

### FIELD OF THE INVENTION

The present invention generally relates to the genetic analysis. More particularly, the present invention relates to computerized and pipelined methods for analyzing genomic data.

### BACKGROUND OF THE INVENTION

Since the completion of the human genome project, technological advances have dramatically increased throughput and decreased the cost of human DNA sequencing, facilitating comprehensive interrogation of coding regions of the genome, transcripts, and whole genome sequences. Studies utilizing this technology have illuminated the underlying genetic basis for rare inherited disease syndromes, refined the molecular understanding of cancer pathogenesis, provided a fine map of rare genetic variation connecting rare variants to common disease risk, and in select cases refined clinical diagnosis and medical therapy.

These initial strides and the continued drop in the cost of high-throughput sequencing have prompted predictions of a new era of medicine personalized to individual genetics. However, downstream interpretation of sequence data remains a formidable barrier to full realization of the promise of genomic medicine. Several applications and data resources exist for predicting the effects of genetic variation on human phenotypes, but there does not yet exist a comprehensive, widely accessible application for clinical interpretation of whole genome sequence data.

### SUMMARY OF THE INVENTION

A methodology is described in embodiment of the present invention for interpretation of genetic and environmental risk in a single participant using a combination of traditional clinical assessment, whole genome sequencing, and integration of genetic and environmental risk factors. An embodiment of the present invention includes an integrated pipeline sequence-to-medical-phenotypes algorithm for interpreting high-throughput human DNA sequencing data. Embodiments of the present invention perform targeted genotyping of clinically relevant variants, rich functional annotation of discovered variants, and prioritize genetic variants according to potential clinical impact, mode of inheritance, and phenotypic presentation. For individual genome sequences, an embodiment of the present invention provides predictive genetic information from personal genomes regarding risk for inherited disease traits and response to pharmacological therapy. For father-mother-child trios, an embodiment of the present invention produces parsimonious fully annotated candidate genetic variants for disease gene discovery and disease diagnosis.

These and other embodiments and advantages can be more fully appreciated upon an understanding of the detailed description of the invention as disclosed below in conjunction with the attached figures.

### BRIEF DESCRIPTION OF THE DRAWINGS

The following drawings will be used to more fully describe embodiments of the present invention.

FIG. 1: Block diagram of a computer system on which embodiments of the present invention can be implemented.

FIG. 2: Flowblock for a method according to an embodiment of the present invention.

FIG. 3: Flowblock for a method according to an embodiment of the present invention.

FIG. 4: Flowblock for a method according to an embodiment of the present invention.

FIG. 5: Flowblock for a method according to an embodiment of the present invention.

### DETAILED DESCRIPTION OF THE INVENTION

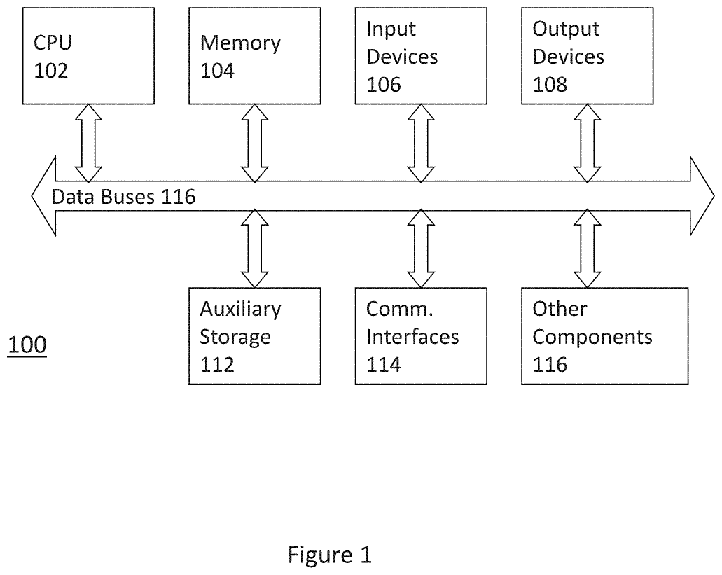

Among other things, the present invention relates to methods, techniques, and algorithms that are intended to be implemented in a digital computer system 100 such as generally shown in FIG. 1. Such a digital computer is well-known in the art and may include the following.

Computer system 100 may include at least one central processing unit 102 but may include many processors or processing cores. Computer system 100 may further include memory 104 in different forms such as RAM, ROM, hard disk, optical drives, and removable drives that may further include drive controllers and other hardware. Auxiliary storage 112 may also be include that can be similar to memory 104 but may be more remotely incorporated such as in a distributed computer system with distributed memory capabilities.

Computer system 100 may further include at least one output device 108 such as a display unit, video hardware, or other peripherals (e.g., printer). At least one input device 106 may also be included in computer system 100 that may include a pointing device (e.g., mouse), a text input device (e.g., keyboard), or touch screen.

Communications interfaces 114 also form an important aspect of computer system 100 especially where computer system 100 is deployed as a distributed computer system. Computer interfaces 114 may include LAN network adapters, WAN network adapters, wireless interfaces, Bluetooth interfaces, modems and other networking interfaces as currently available and as may be developed in the future.

Computer system 100 may further include other components 116 that may be generally available components as well as specially developed components for implementation of the present invention. Importantly, computer system 100 incorporates various data buses 116 that are intended to allow for communication of the various components of computer system 100. Data buses 116 include, for example, input/output buses and bus controllers.

Indeed, the present invention is not limited to computer system 100 as known at the time of the invention. Instead, the present invention is intended to be deployed in future computer systems with more advanced technology that can make use of all aspects of the present invention. It is expected that computer technology will continue to advance but one of ordinary skill in the art will be able to take the present disclosure and implement the described teachings on the more advanced computers or other digital devices such as mobile telephones or “smart” televisions as they become available. Moreover, the present invention may be implemented on one or more distributed computers. Still further, the present invention may be implemented in various types of software languages including C, C++, and others. Also, one of ordinary skill in the art is familiar with compiling software source code into executable software that may be stored in various forms and in various media (e.g., magnetic, optical, solid state, etc.). One of ordinary skill in the art is familiar with the use of computers and software languages and, with an understanding of the present disclosure, will be able to implement the present teachings for use on a wide variety of computers.

The present disclosure provides a detailed explanation of the present invention with detailed explanations that allow one of ordinary skill in the art to implement the present invention into a computerized method. Certain of these and other details are not included in the present disclosure so as not to detract from the teachings presented herein but it is understood that one of ordinary skill in the art would be familiar with such details.

### Introduction

Several applications and data resources exist for predicting the effects of individual nucleotide substitutions. These can be broadly classified into one of two categories: 1) resources that provide predictions about pathogenicity of genetic variants in absence of human genotype-phenotype association data, and 2) resources that seek to apply previous human genotype-phenotype association data to re-sequencing (or other genotype) data. The first group of resources utilizes characteristics of amino acid substitutions, putative change in protein secondary structure, disruption of known structural motifs, and several derivative metrics of evolutionary conservation and constraint to provide predictions about pathogenicity of single nucleotide substitutions. The second group of resources links data from previous linkage studies, family co-segregation data, genome wide association scans, and other sources into searchable databases of genotype-phenotype associations.

The first set of resources, while suited to annotation of individual variants with no human genotype-phenotype association data, ignores the large repository of data connecting genetic variation with human disease phenotypes, and provides no clinical context for interpretation of variant annotations. The second set of resources is not well suited to annotation of comprehensive re-sequencing data, contains annotation errors and common polymorphisms, by some estimates comprising approximately >25% of the entries, and is contaminated by descriptions of trait susceptibility loci of questionable clinical relevance.

There does not yet exist, to date, a readily available pipeline for comprehensive clinical interpretation of sequence variants that integrates predicted methods and the bulk of published disease-genotype associations for genetic assessment of inherited disease risk and predicted drug response. Embodiments of the present invention address certain of these shortcomings of the prior art.

### Sequence to Medical Phenotypes

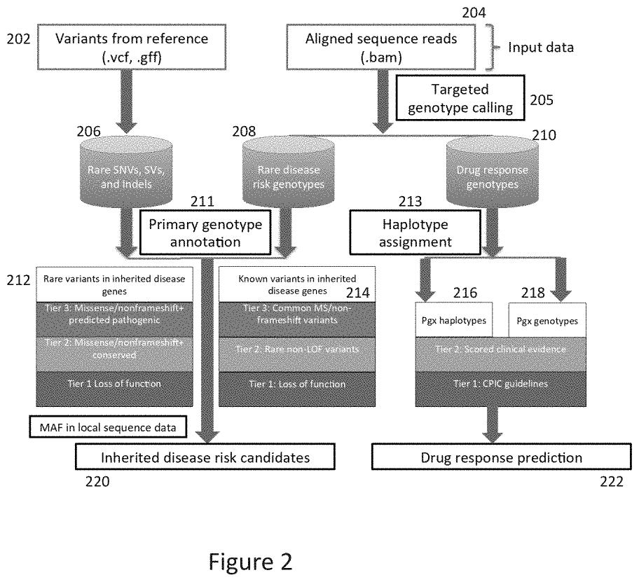

Shown in FIG. 2 is a block diagram of a method according to an embodiment of the present invention generating sequence-to-medical-phenotypes information. As shown, at steps 202 and 204 digitized genomic information is received such as may be generated from a primary sequence analysis. Among other things, sequence alignment and variant identification information is received. For example, in an embodiment of the present invention, at step 202, variants from a reference are received in the form of .vcf or .gff files. The .vcf file is generally used to input variant call information but other similar files are appropriate as they may exist or be developed. The .gff file is generally used to provide general feature information about genes and other features of DNA, RNA and protein sequences but other similar files are appropriate as they may exist or be developed. Also, binary alignment information is received in the form of a .bam file. Information that can be received as input includes variant call files for single nucleotide variants and indels (SNVs, indels) and, optionally, genomic feature format files for structural variants (SVs). Other information as it may exist or may be subsequently developed can be input as would be known to those of ordinary skill in the art upon and understanding of the present disclosure. This received input is then referenced against rare SNVs, SVs, and indels 206.

It should be noted that embodiments of the present invention are shown and described in a pipeline fashion that is amenable to efficient pipeline computerized processing. For example, certain of the steps to be described may be independent of other steps such that they can be performed independently on different processors or processing cores. Moreover, embodiments of the present invention balance the overall methods among various coprocessors. In this way, as additional computational needs are required for certain steps, more processors or processing cores can be dedicated to such steps. Those of ordinary skill in the art will understand that there are many ways of balancing computational resources in a pipeline architecture such as described in embodiment of the present invention.

At step 205, targeted genotype calling is performed. In an embodiment of the present invention, targeted genotype calling is performed with reference to rare disease risk genotypes 208 and drug response phenotypes 210. In another embodiment of the present invention, the computerized task for targeted genotype calling is mapped to a plurality of processors among available processors. The assignment of the number of processors can, for example, be specified through a command line interface, a graphical interface, or through an input file.

In an embodiment of the present invention, a GATK haplotype caller is used for indels and a unified genotype caller for SNVs. In an embodiment, call genotypes are made from the BAM file (if specified) for known rare disease risk variants and drug response variants using, for example, the Human Gene Mutation Database variants in Clinvar and pharmGKB, respectively.

In an embodiment of the present invention, calls are filtered using hard filtering based on, for example, quality, depth, mapping quality, and allele balance. In yet another embodiment, calls are re-annotated to add dbsnp identifiers.

Primary genotype annotation is then performed at step 211 using the results from the queries of rare SNvs, SVs, and indels 206 and rare disease risk genotypes 208. For example, in an embodiment of the present invention, genotype annotation is performed for all variants from the received reference at step 202. In such an embodiment, a .vcf file is used along with an optional .gff input file.

In an embodiment of the present invention, as part of primary genotype annotation 211, a conversion is made to annovar file input format. Conversion to such format facilitates the use of certain computerized methods for functionally annotating genetic variants detected from diverse genomes. For example, with a list of variants with chromosome, start position, end position and observed nucleotides, computerized methods using annovar can identify whether SNPs or indels cause protein coding changes among other things. As noted previously, embodiments of the present invention lend themselves to pipelined processing and processing among various processors or cores. For example, here, individual annovar annotation processes are mapped to a pool of n processes.

In an embodiment of the present invention, intermediate annotation files are created for each of the received datasets (e.g., SNVs, SVs, and indels). Also, the plurality of individual annotations files is collected and reduced to two files (e.g., *.allvars.genome_summary.tsv, and .clinvar.genome_summary.tsv, representing annotated variants from reference and annotated known rare disease risk genotypes) for SNVs/indes1 and one file for SVs (e.g., *.sys.genome_summary.tsv). Such files can be, for example, text files in a tab separated format.

Rare variants in inherited disease genes are then identified at step 212. For example, in an embodiment of the invention, rare variants in inherited disease genes are categorized into a plurality of tiers. For example, as shown in FIG. 2, Tier 1 represents rare loss of function variants, where rarity is generally defined as variants with the highest allele frequency below user-defined threshold in user defined population background (e.g., CEU, Caucasian, CHB/JPT, East Asian, AFR, African). Tier 2 represents rare missense or non-frameshift indels affecting nucleotides with consensus evidence by both algorithms considered for evolutionary conservation/constraint according to the following values cataloged in the database of nonsynonymous functional predictions28: GERP++ score>2; PhyloPnew>0.95. Tier 3 represents rare missense or non-frameshift indels with predicted pathogenicity by three or more algorithms according to the following definitions as cataloged in the database of nonsynoymous functional predictions28: SIFT, “Damaging;” LRT, “Deleterious;” PolyPhen2, “Probably damaging” or “Possibly damaging;” Mutation taster, “Disease causing automatic” or “disease causing.” In an embodiment, outputs from step 212 are included as information for inherited disease risk candidates 220. In an embodiment not shown in FIG. 2, Tier 4 represents all rare variants not meeting criteria for Tiers 1-3.

As a final filter for both previously reported variants in monogenic disease genes and previously unreported, rare variants in monogenic disease genes, an embodiment of the present invention uses catalogs of local allele frequency and genotype information to exclude variants that are observed more frequently than would be expected in the local sequencing environment (according to user defined criteria). This filter serves to identify and exclude variants whose previously unappreciated high allele frequency is a result of false negatives in population genetic surveys or systematic false positives specific to the sequence assessment pipeline. Similar filters have proven effective in excluding such systematic artifacts in other contexts.

As would be known to those of ordinary skill in the art upon an understanding of the present invention, the criteria for the various tiers can be modified to meet particular needs that may arise in different situations.

Known variants in inherited disease genes are identified at step 214. For example, in an embodiment of the present invention, known variants in inherited disease genes are categorized into a plurality of tiers. For example as shown in FIG. 2, Tier 1 represents loss of function variants (e.g., splice dinucleotide disrupting, nonsense, nonstop, and frameshift indels). Tier 2 represents rare variants cataloged in HGMD, regardless of functional annotation. Rarity is generally defined as transcription factor MAF no greater than 1% in any of the following population genetic surveys: ethnically-matched population in HapMap 2 and 3, the 1000 genomes phase 1 data27 from an ethnically-matched super population, and global allele frequency, the 1000 genomes pilot 1 project global allele frequency, 69 publicly available genomes released by Complete Genomics, and the NHLBI Grand Opportunity exome sequencing project global allele frequency. Tier 3 represents all non-rare missense and non-frameshift indels. In an embodiment, outputs from step 212 are included as information for inherited disease risk candidates 220. In an embodiment not shown in FIG. 2, Tier 4 represents all rare variants not meeting criteria for Tiers 1-3.

As would be known to those of ordinary skill in the art upon an understanding of the present invention, the criteria for the various tiers can be modified to meet particular needs that may arise in different situations.

In an embodiment of the present invention, rare SVs in inherited disease genese (e.g., genes specified by a user or in an clinvar default) are filtered by a predetermined allele frequency in allele frequency databases using a rarity criteria as discussed above. In an embodiment, these criteria are user specified. Information is retained if it is predicted to cause exonic disruption (e.g., annotated as exonic) of inherited disease genes. In an embodiment, user input information (e.g., user input or input files) identifies inherited disease genes for individuals.

Haplotype assignment is then performed at step 213 as shown in FIG. 2 using the targeted genotypes of steps 205/208/210. For example, in an embodiment to the present invention, star allele haplotype assignment is performed as follows. Skeleton haplotype pairs are created using all confidently identified homozygous SNVs and indels. The full set of complementary haplotypes is then generated using heterozygous variant calls. In an embodiment, a perfect match search is performed for each haplotype and its complement among the described haplotypes defining the known “star” alleles associated with clinical drug response. In an embodiment, if a perfect match is not found, the set of possible haplotype pairs is given but no star allele assignment is attempted. Also, if more than one pair of possible star alleles is found matching possible haplotypes generated from WGS data, all possible star allele combinations are reported. An embodiment does not provide haplotype resolution beyond that suggested by the confidently called genotypes.

As shown in FIG. 2, pharmacogenomics haplotypes and genotypes are identified at steps 216 and 218, respectively. For example, in an embodiment of the invention, pharmacogenomics haplotypes and genotypes are categorized into a plurality of tiers. For example, as shown in FIG. 2, Tier 1 represents haplotype and genotype assignments per CPIC (Clinical Pharmacogenetics Implementation Consortium) dosing guidelines. In an embodiment, an analysis is performed for every gene haplotype definition map in a collection (e.g., a resource folder). In an embodiment, using star alleles called in Haplotype Assignment 213 set, merge diplotypes (haplotype pairs) with CPIC annotations, providing drug disease and administration guidelines for simvastatin, warfarin, clopidogrel, thiopurines, and codeine (e.g., a default condition) or more comprehensive annotations as updated in a resource folder. In an embodiment, the output of Tier 1 includes drug/haplotype combinations for a predetermined set (e.g., as contained in a resource folder). Tier 2 represents Pgx haplotypes and genotypes meeting predetermined scored clinical evidence. For example, using targeted genotypes identified in Targeted Genotype calling steps 205/208/210 above, information is merged with PharmGKB clinical annotations (default) or used with provided annotations as updated in a resource folder, for example, to provide drug response predictions for individual variant genotypes scored by level of clinical evidence, type of association, drug, association, ethnic background. In an embodiment, the output for Tier 2 includes dosage, efficacy, and toxicity information. In still another embodiment, other information can be identified for output. The collection of the results of Tiers 1 and 2 provide drug response prediction information 222 as shown in FIG. 2.

Embodiments of the present invention find wide application in genomics. For example, applications for embodiments of the present invention include: 1) Identification of medically actionable genetic variants with disease risk implications in pre-symptomatic individuals in WGS data; 2) Identification of genetic variants in WGS associated with drug response; and 3) Identification of genetic variants in WGS associated with disease in families and individuals.

Embodiments of the present invention present advantages over the prior ar. For example, embodiments of the present invention include fully integrated sets of methods for clinical interpretation of whole genome sequence data. Also, the present invention provides methods for automated haplotype identification and comprehensive annotation of drug response predictions from whole genome sequence data. Embodiments of the present invention provide methods for identifying apparent de novo mutations in father-mother-child trios while excluding systematic artifacts. Also, embodiments seamlessly integrate with a variety of variant call outputs and sequence maps. Embodiments also provide reference-agnostic disease risk prediction and provide comprehensive disease risk prediction and drug response prediction using efficient and fast methods and data sources for haplotype determination and annotation.

### Alternative Embodiments

Alternative embodiments of the present invention are shown in FIGS. 1-3. As will be described below. As will be evident, discussions of FIGS. 1-3 are applicable to FIG. 2 and vice versa.

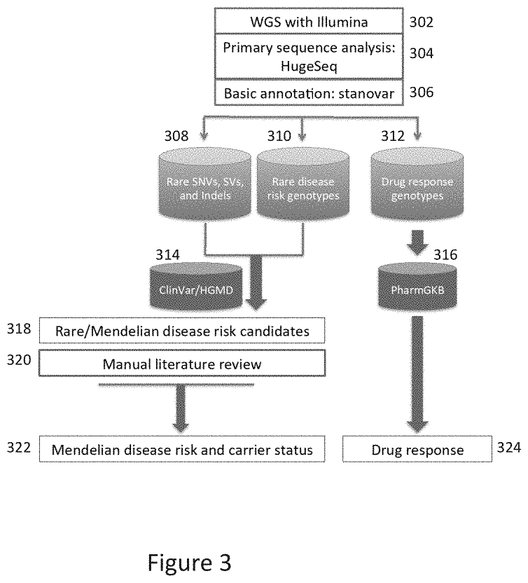

Methods for clinical interpretation in the context of disease gene finding in inherited disease syndromes and predictive genetic variant annotation are shown in FIG. 3. For example, in an embodiment of the present invention, whole genome sequencing is performed using an Illumina tool at step 302. Other methods can also be used at step 302 as known to those of ordinary skill in the art. A primary sequence analysis using HugeSeq is performed at step 304. Other methods can also be used at step 304 as known to those of ordinary skill in the art. A basic annotation (e.g., stanovar) is performed at step 306. Other methods can also be used at step 306. The information from step 306 (as well as 304 and 302) is then referenced against rare SNVs, Svs and indels 308, rare disease risk genotypes 310, and ClinVar/HGMD 314 information to generate rare/Menelian disease risk candidates at step 318. In an embodiments, the rare/Mendelian disease risk candidates are then used in conjunction with the results of a manual literature review to generate Mendelian disease risk and carrier status information at step 322. In a pipelined manner, the information from step 306 (as well as 304 and 302) is then referenced against drug response genotypes 312 and PharmaGKB 316 information to generate drug response information 324.

It should be noted that embodiments of the present invention are shown and described in a pipeline fashion that is amenable to efficient pipeline computerized processing. For example, certain of the steps to be described may be independent of other steps such that they can be performed independently on different processors or processing cores. Moreover, embodiments of the present invention balance the overall methods among various coprocessors. In this way, as additional computational needs are required for certain steps, more processors or processing cores can be dedicated to such steps. Those of ordinary skill in the art will understand that there are many ways of balancing computational resources in a pipeline architecture such as described in embodiment of the present invention.

Discovery of Variants from an Ethnically-Matched Reference Sequence and Genotyping of Clinically Relevant Variants

High-throughput re-sequencing with current sequencing platforms requires a reference genome for sequence assembly and variant identification. The reference genome that is currently used for alignment of human re-sequencing data and variant identification (the NCBI reference genome) is derived from a collection of DNA samples from a small number of anonymous volunteers. It is currently the only “finished-grade” human genome in that it was assembled de-novo from long sequence reads and covers approximately 99% of the euchromatic human genome. However, it represents a small sampling of human genetic variation. As such, at approximately 1.6 million genomic positions, the NCBI reference sequence differs from the major allele in each of the three Haplotype Map (HapMap) populations, and at approximately 800,000 positions, the minor allele for all three population groups is represented on the NCBI reference sequence. These minor alleles span approximately 4,500 variant positions associated with common complex disease traits.

To address these issues, a major allele reference sequence for each of the three major HapMap populations was developed. This is utilized it in mapping short read data and variant identification using high-throughput sequencing. Application of this approach to variant identification in whole genome sequence data from a nuclear family of four reduced genotype error by approximately 40% at disease-associated variant loci and resulted in greater concordance with genotype calls from orthogonal genotyping technologies.

In an embodiment of the present invention, the methods of the present invention can use major allele reference genomes if the ethnicity of study participants is known. In an alternative embodiment, it may be preferable to use standard (or SNP-masked) standard reference sequences if ethnicity is not known or if performing joint genotype calling of a multi-ethnic study cohort. In the latter case, alignment can be performed using an ethnicity-specific major allele reference to maximize sensitivity of short read alignment, and a standard or SNP-masked standard reference sequence may be used for variant and genotype identification, thereby standardizing the reference allele in the variant call file.

There are well-document disease risk and drug response alleles in the reference sequence, most notably Factor V Leiden allele associated with hereditary thrombophilia. Comparison of genome sequence data that is homozygous for these alleles to this reference sequence will naturally not produce a variant call. This issue is partially addressed by the use of major allele reference sequences. A more comprehensive approach is to perform targeted genotype calling of all loci considered to be of phenotypic importance. This approach is reference agnostic up to reference bias associated with short read alignment and mapping. Embodiments of the present invention use input interval call files representing previously reported Mendelian disease associated loci and loci associated with drug response to provide targeted genotype calls, irrespective of reference base, using the GATK Unified Genotyper (for SNVs) and Haplotype Caller (for indels). Embodiments of the present invention also provide metrics for coverage of loci with known importance to human health and disease, thereby providing confidence that a disease-associated allele is indeed not present, rather than just under-sequenced or otherwise not confidently ascertained. As compared with methods that store diploid calls for all reference genomic positions, the approach to genotype interrogation facilitates downstream variant annotation while minimizing storage requirements for variant data. To facilitate updated genotype identification as new loci of relevance to human health and disease are discovered, binary alignment (BAM) files from the secondary sequence analysis are retained for future analysis.

Annotation of Genetic Variants with Respect to Functional Genetic and Clinical Phenotypes

Rich functional genomic annotation is a prerequisite to sequence interpretation pipelines that aim to provide testable biological hypotheses about the basis for described disease syndromes and for disease risk and drug response prognostication. The annovar framework was extended to provide rich gene-based, functional genomic, regulatory, allele frequency, and phenotypic annotation. This basic annotation pipeline, stanovar, provides 94 annotations for SNVs and indels in .vcf format (Table 1 attached as Appendix A) and 39 annotations for SVs in .gff format (Table 2 attached as Appendix B).

An embodiment of the present invention can also leverage gene coexpression network topology information to provide quantitative prior expectations about gene-level pathogenicity for contextualizing individual variation data. For example, output of an embodiment of the present invention can be used to identify genetic variation occurring in genes that are coexpressed with known disease genes, thereby implicating by association variants perturbing certain network topologies. In an embodiment, the default module comes pre-loaded with gene coexpression network topology representing gene expression microarray data from 75 normal, unused human donor hearts, tissue from 49 human hearts with right- or left-ventricular hypertrophy, and 436 explanted human hearts with dilated cardiomyopathy. Disease-specific topologies can be used to assess dynamic gene-gene interactions that are context specific.

### Bioinformatic Prioritization of Candidate Mendelian Disease Risk Variants

While rich annotation of genetic variants and genotypes with potential clinical significance is a pre-requisite for downstream analysis, prioritization of variants for manual review, in the case of individual genome interpretation, or intersection with inheritance state information, in the case of disease gene identification projects, is arguably the most crucial part of the analytical pipeline. An embodiment of the present invention uses a prioritization heuristic that at once provides a parsimonious set of candidates for manual review and a comprehensive assessment of previously reported genetic variation.

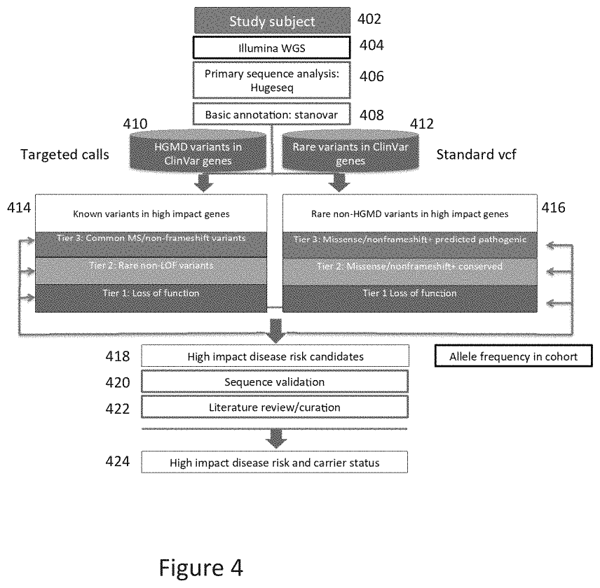

The overall heuristic for prioritization of previously reported variants in monogenic disease genes, as well as rare and novel variants in monogenic disease genes with no previously reported phenotypic association, is described in FIG. 4. For example, in an embodiment of the present invention, whole genome sequencing is performed using an Illumina tool at step 404 for a study subject 402. Other methods can also be used at step 402 as known to those of ordinary skill in the art. A primary sequence analysis using HugeSeq is performed at step 406. Other methods can also be used at step 406 as known to those of ordinary skill in the art. A basic annotation (e.g., stanovar) is performed at step 408. Other methods can also be used at step 408 as known to those of ordinary skill in the art. The information from step 408 (as well as 406 and 404) is then referenced against HGMD variants in ClinVar genes 410 to generate candidates for known variants in high impact genes at step 414. In an embodiments, the known variants in high impact genes are categorized into tiers (e.g., Tiers 1-3) in a similar manner as discussed above. For example, in an embodiment, Tier 1 represents loss of function candidates (e.g., (splice dinucleotide disrupting, nonsense, nonstop, and frameshift indels). Tier 2 represents rare non-loss-of-function variants. In an embodiment, Tier 2 represents rare variants cataloged in HGMD, regardless of functional annotation. Rarity is defined as MAF no greater than 1% (default, can use alternative cut-offs using optional flags in command line) in any of the following population genetic surveys: ethnically-matched population in HapMap 2 and 3, the 1000 genomes phase 1 data27 from an ethnically-matched super population, and global allele frequency, the 1000 genomes pilot 1 project global allele frequency, 69 publicly available genomes released by Complete Genomics, and the NHLBI Grand Opportunity exome sequencing project global allele frequency. Tier 3 represents common MS/non-frameshift variants. For example, Tier 3 may be all non-rare missense and non-frameshift indels. In an embodiment, Tier 4 (not shown) represents all variants not meeting criteria for Tiers 1-3.

The information from step 408 (as well as 406 and 404) is also referenced against rare variants in ClinVar genes 412 to generate candidates for rare non-HGMD variants in high impact genes at step 416. In an embodiments, the rare non-HGMD variants in high impact genes are categorized into tiers (e.g., Tiers 1-3) in a similar manner as discussed above. For example, in an embodiment, Tier 1 represents loss of function candidates; Tier 2 represents missense/nonframeshit conserved variants, and Tier 3 represents missense/nonframeshift predicted pathogenic variants. In an embodiment, Tier 4 (not shown) represents all variants not meeting criteria for Tiers 1-3.

The results form steps 414 and 416 are then collected at step 418 as high impact disease risk candidates. A sequence validation is performed at step 420 in an embodiment of the present invention. The resulting candidates are then used in conjunction with the results of a manual literature review at step 422 to generate high impact disease risk and carrier status information at step 424.

It should be noted that embodiments of the present invention are shown and described in a pipeline fashion that is amenable to efficient pipeline computerized processing. For example, certain of the steps to be described may be independent of other steps such that they can be performed independently on different processors or processing cores. Moreover, embodiments of the present invention balance the overall methods among various coprocessors. In this way, as additional computational needs are required for certain steps, more processors or processing cores can be dedicated to such steps. Those of ordinary skill in the art will understand that there are many ways of balancing computational resources in a pipeline architecture such as described in embodiment of the present invention.

In an embodiment, a default mode first reduces all variants to a set that occurs within 2,725 genes cataloged in ClinVar (2,716 in females due to cataloged disease associations within nine genes on the Y chromosome), manually curated to exclude drug response associations and common disease susceptibility loci. Alternatively, gene sets can be provided by the user and utilized for genetic variant filtering based on the phenotypic features of the disease queried.

Previously reported variants in disease mutation catalogs include a significant number of common polymorphisms, mapping errors, legacy coordinates, and common disease susceptibility loci that are unlikely to be relevant to monogenic disease risk. Thus, an embodiment of the present invention prioritizes variants previously reported in the Human Gene Mutation Database (HGMD) first by expected pathogenicity and next by allele frequency. Previously reported variants cataloged in HGMD are separated into four tiers of potential pathogenicity:

Tier 1: Loss of function variants (splice dinucleotide disrupting, nonsense, nonstop, and frameshift indels).

Tier 2: All rare variants cataloged in HGMD, regardless of functional annotation. Rarity is defined as MAF no greater than 1% (default, can use alternative cut-offs using optional flags in command line) in any of the following population genetic surveys: ethnically-matched population in HapMap 2 and 3, the 1000 genomes phase 1 data27 from an ethnically-matched super population, and global allele frequency, the 1000 genomes pilot 1 project global allele frequency, 69 publicly available genomes released by Complete Genomics, and the NHLBI Grand Opportunity exome sequencing project global allele frequency.

Tier 3: All non-rare missense and non-frameshift indels.

Tier 4: All variants not meeting criteria for tiers 1-3.

Variants meeting criteria for tiers 1-3 are retained for downstream manual review in the case of individual genome interpretation, and for intersection with inheritance state information in the case of disease-targeted analyses. As the expected allele frequency of potentially pathogenic variants is likely to vary greatly with disease prevalence, penetrance, and mode of inheritance, allele frequency filters are not used for tiers one and three, thereby allowing for prioritization of functional alleles with previously described disease associations that would not otherwise pass strict allele frequency filters, for instance the deltaF508 allele in CFTR or the Factor V Leiden allele.

Rare and private variation, as a result of recent population expansion and purifying selection, continues to constitute a significant proportion of human genetic variation, even in large surveys of individual variation. Some of these rare and private alleles will have monogenic disease risk and carrier status relevance, and therefore an embodiment of the present invention also prioritizes select previously unreported, but potentially pathogenic, rare and novel variants in monogenic disease genes, incorporating consensus evidence for evolutionary constraint/conservation and pathogenicity prediction. These computational methods for scoring genetic variants have, in isolation, modest classification accuracy and inter-algorithm concordance. Approaches to rating the potential pathogenicity of variants based on consensus of commonly used prediction algorithms have been shown to have superior calibration and discriminative accuracy when compared with individual predictions. An embodiment of the present invention imposes a prior expectation that such alleles are more likely to occur in genes in which previously reported variants have produced Mendelian disease phenotypes, but also archives and categorizes all other variants for review and potential reclassification as genetic knowledge expands, or for intersection with inheritance state data. Rare (defined as above) and novel variants with no previously described phenotype association in monogenic disease genes are prioritized into four tiers of potential pathogenicity in an embodiment of the present invention:

Tier 1. Rare loss of function variants, defined as above.

Tier 2: Rare missense or non-frameshift indels affecting nucleotides with consensus evidence by both algorithms considered for evolutionary conservation/constraint according to the following values cataloged in the database of nonsynonymous functional predictions: GERP++ score>2; PhyloPnew>0.95.

Tier 3: Rare missense or non-frameshift indels with predicted pathogenicity by three or more algorithms according to the following definitions as cataloged in the database of nonsynoymous functional predictions: SIFT, “Damaging”; LRT, “Deleterious”; PolyPhen2, “Probably damaging” or “Possibly damaging”; Mutation taster, “Disease causing automatic” or “disease causing”.

Tier 4: All rare variants not meeting criteria for tiers 1-3.

Annotation of Personal Genetic Variation Associated with Drug Response

Predicting drug response from WGS data requires generation of best-guess haplotypes from short-read sequence data for which haplotype phase is often not determined molecularly. An embodiment of the present invention produces best-guess haplotype pairs from confidently genotyped SNVs and indels identified as above. To do this embodiment first creates skeleton haplotype pairs using all confidently identified homozygous SNVs. The full set of complementary haplotypes is then generated using heterozygous variant calls. A perfect-match search is performed for each haplotype and its complement among described haplotypes defining the known “star” alleles associated with clinical drug response. If a perfect match is not found, the set of possible haplotype pairs is given but no star allele assignment is attempted. If more than one pair of possible star alleles is found matching possible haplotypes generated from WGS data, all possible star allele combinations are reported.

An embodiment of the present invention does not provide haplotype resolution beyond that suggested by the confidently called genotypes. That is, if a variant is not confidently called, haplotypes that are uniquely defined by these “tags” variants are not disambiguated from other possible star allele-defining haplotypes, and a set of possible star alleles corresponding to each reduced haplotype and its conjugate are reported. The haplotype determination is purposefully designed to only give high-confidence predictions, leaving the task of disambiguating star alleles in the setting of uncertain genotype calls or uncommon haplotypes to a human curator.

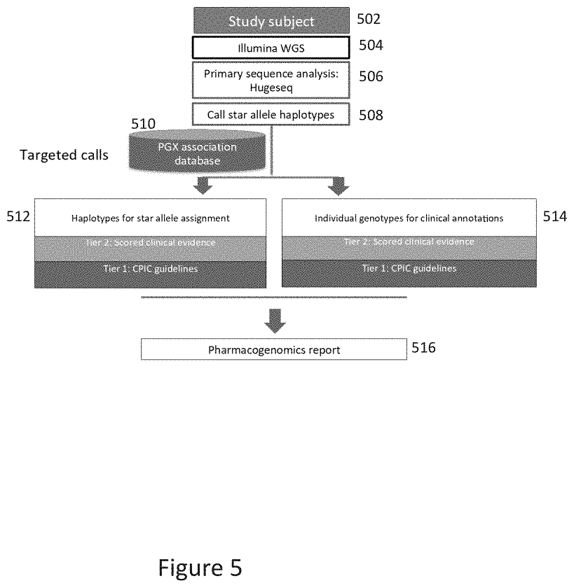

Another embodiment also annotates and reports single variant drug response associations cataloged in the PharmGKB knowledgebase at a level of evidence (for definitions, see http://www.pharmgkb.org/page/clinAnnLevels) defined by the user as shown in the embodiment of FIG. 5. For example, in an embodiment of the present invention, whole genome sequencing is performed using an Illumina tool at step 504 for a study subject 502. Other methods can also be used at step 504 as known to those of ordinary skill in the art. A primary sequence analysis using HugeSeq is performed at step 506. Other methods can also be used at step 506 as known to those of ordinary skill in the art. A call of star allele haplotypes is performed at step 508. Other methods can also be used at step 508 as known to those of ordinary skill in the art. The information from step 508 (as well as 506 and 504) is then referenced against a PGX association database at step 510 to generate candidates for haplotypes for star allele assignment at step 512. In an embodiments, the haplotypes for star allele assignment are categorized into tiers (e.g., Tiers 1 and 2) in a similar manner as discussed above. For example, in an embodiment, Tier 1 represents candidates per CPIC guidelines; Tier 2 represents those candidates with scored clinical evidence of a predetermined value. The information from step 508 (as well as 506 and 504) is also referenced against a PGX association database at step 510 to generate candidates for individual genotypes for clinical annotations at step 514. In an embodiment, the individual genotypes for clinical annotations are categorized into tiers (e.g., Tiers 1 and 2) in a similar manner as discussed above. For example, in an embodiment, Tier 1 represents candidates per CPIC guidelines; Tier 2 represents those candidates with scored clinical evidence of a predetermined value. The results form steps 512 and 514 are then collected at step 516 into a pharmacogenics report.

It should be noted that embodiments of the present invention are shown and described in a pipeline fashion that is amenable to efficient pipeline computerized processing. For example, certain of the steps to be described may be independent of other steps such that they can be performed independently on different processors or processing cores. Moreover, embodiments of the present invention balance the overall methods among various coprocessors. In this way, as additional computational needs are required for certain steps, more processors or processing cores can be dedicated to such steps. Those of ordinary skill in the art will understand that there are many ways of balancing computational resources in a pipeline architecture such as described in embodiment of the present invention.

Disease Associated Variant Discovery in Father-Mother-Child Trios with Simplex Phenotypes

Genome-wide genetic interrogation in father-mother-child trios with apparently simplex phenotypes can be a powerful tool for genetic association discovery. Classically these investigations are performed using discrete filtering to identify apparent de novo, compound heterozygous, and rare homozygous mutations. De novo mutations are typically discovered via searching for Mendelian inheritance abnormalities (MIAs) that are consistent with the segregation of phenotypes within the family. Discrete filtering is encumbered by several challenges, however. First, the true per-generation de novo mutation rate is two to three orders of magnitude lower than the sequence error rate using current high throughput sequencing technology. In addition to stochastic error modes, there are systematic error modes arising from sequences in the human reference genome that are compressed relative to common repetitive sequences, low complexity and GC and homopolymer rich regions, and other regions of the human genome that are problematic to accurately sequence, align, or genotype. Roach, et al, developed an HMM-based classifier to identify these regions in family quartets and also to provide relative inheritance state information for WGS. An embodiment of the present invention utilizes a simplified HMM classifier that bins WGS or WES data into one of three categories (three hidden states): “good data”, “compression”, or “MIA-rich” regions. The latter two categories represent variant data that is highly likely to contain systematic artifact and can be excluded from downstream analysis and-or interpretation. An embodiment writes this information in the “Filter” field of standard vcf output, allowing for soft-filtering or manual inspection of these regions. An embodiment uses chromosomal distance between variant markers as a prior expectation in the HMM, thereby facilitating for the first time the use of this HMM-based approach in WES, which by virtue of sequence capture is sparse outside targeted regions and dense within targeted regions.

Second, apparently simplex disease phenotypes can arise from a variety of possible underlying genetic architectures and modes of inheritance, and pre-specifying one mode can lead to a lack of sensitivity. To address this, once HMM-based regional classification has been performed, an embodiment of the present invention will output 1) apparent de novo events, 2) all instances of compound heterozygosity in a gene in which at least one variant in the pair is rare according to user specific criteria 3) rare homozygous mutations, and 4) instances of apparent hemizygosity which are candidates for loss of function, 5) rare variants in known inherited disease genes fitting an autosomal dominant inheritance model with reduced penetrance. This output can be used for manual inspection in single trio studies or as a prior expectation for gene regions fed forward into collapsing statistics if a cohort of trios is studied. In the latter case an embodiment leverages inheritance information to reduce the number of gene regions queried and thus the number of statistical comparisons performed between case and control cohorts.

### Inputs, Implementation and Parallelization

An embodiment of the present invention accepts as required input a vcf file prepared by Illumina Isaac, GATK, the Complete Genomics variant discovery pipeline, or Real Time Genomics. Optional inputs include 1) binary alignment map file, required for targeted genotype calling and annotation of known inherited disease risk alleles and pharmacogenomic annotation, 2) genome feature format file describing structural variant events, 3) local site frequency spectrum for filtering of inherited disease risk candidates. In trio mode, an embodiment requires jointly called genotypes and sample identifiers for father, mother, and child. Initial annotation of genetic variants for gene, functional genomic, and clinical phenotypes (stanovar) is performed using python/perl and parallelized using the “multiprocessing” module in python. Processing time for stanovar scales roughly as 1/n, where n is the number of processors allocated to the task. An embodiment is implemented in cython/python/shell and also parallelized using the “multiprocessing” modules in python.

Other embodiments of the present invention include but are not limited to:
Extension of father-mother-child analysis to larger family pedigrees
Integration of common disease risk prediction from large genome wide association studies
Integration with variant identification pipelines
Integration of other molecular signature data, such as genotype arrays, gene expression microarrays, and RNA sequencing

Population wide sequencing is a near-term reality in medicine, and theability to interpret genome sequences from individuals will determine the commercial viability of this technology for advancing personalized health care. With these genome interpretation methods as disclosed herein, a solution is presented for rich genome annotation, clinical genotype discovery, and identification of clinically important disease risk and drug response predictions in whole genome sequence data.

Further details about embodiments of the present invention are included in Tables 1 and 2 included in the Appendixes A and B, respectively.

It should be appreciated by those skilled in the art that the specific embodiments disclosed herein may be readily utilized as a basis for modifying or designing other algorithms or systems. It should also be appreciated by those skilled in the art that such modifications do not depart from the scope of the invention as set forth in the appended claims. For example, variations to the methods can include changes that may improve the accuracy or flexibility of the disclosed methods.

### Appendix A

| Field | Category | Description |
|---|---|---|
| function | gene | variant function according to UCSC knowngene |
| gene_knowngene | gene | gene according to UCSC knowngene |
| exonicFunction_knowngene | gene | exonic function according to UCSC knowngene |
| aaChange_knowngene | gene | amino acid change according to UCSC knowngene |
| exonicFunction_gencode | gene | exonic function according to Encode/GeneCode |
| | | V14/Ensembl(deprecated) gene annotation, all transcripts |
| aaChange_gencode | gene | amino acid change according to Encode/GeneCode |
| | | V14/Ensembl(deprecated) gene annotation, all transcripts |
| exonicFunction_refgene | gene | exonic function according to refseq |
| aaChange_refgene | gene | amino acid change according to refseq |
| pfamA_domain | gene | pfam level A annotations (manually curated) for protein |
| | | family/domain: http://pfam.sanaer.ac.uk/ |
| bioCyc_id | gene | bioCyc identifier http://biocyc.org |
| bioCyc_pathway | gene | bioCyc pathway http://biocyc.org |
| kegg_id | gene | KEGG id |
| kegg_pathway | gene | KEGG pathway |
| pseudogene_id | gene | is variant in a pseudogene according to Yale Pseudogene |
| | | database 70, pseudogene id |
| pseudogene_class | gene | is variant in a pseudogene according to Yale Pseudogene |
| | | database 70, pseudogene class |
| has_pseudogene_eValue | gene | is variant in a gene with sequence similarity to a known |
| | | pseudogene, according to ensembl/Yale intersection, blast e |
| | | value, 0-1 |
| has_pseudogene_name | gene | is variant in a gene with sequence similarity to a known |
| | | pseudogene, according to ensembl/Yale intersection, |
| | | pseudogene name |
| encode_dnaseCluster | regulation | encode DNAse hypersensitivity cluster score, 0-1000 |
| encode_tfbsChip_score | regulation | encode TFBS CHIP score, 0-1000 |
| encode_tfbsChip_name | regulation | encode TFBS CHIP factor name |
| encode_HMMgm12878 | regulation | HMM-based classification of regulatory regions in |
| | | lymphoblastoid cells from NA12878 |
| encode_HMMhesc | regulation | HMM-based classification of regulatory regions IH1-hESC |
| encode_HMMhsmm | regulation | HMM-based classification of regulatory regions in skeletal |
| | | muscle myoblasts |
| encode_HMMhuvec | regulation | HMM-based classification of regulatory regions in umbilical |
| | | vein endothelial cells |
| encode_HMMnhek | regulation | HMM-based classification of regulatory regions in epidermal |
| | | keratinocytes |
| encode_HMMnhlf | regulation. | HMM-based classification of regulatory regions in lung |
| | | fibroblasts |
| cpg_Island | regulation | CpG islands, cpg number |
| regulome_cat1_annotation | regulation | Category 1 regulome variant annotation from dbsnp132: |
| | | http://regulome.stanford.edu/ |
| regulome_cat1_ class | regulation | Category 1 variant evidence class: |
| | | http://genome.cshlp.org/content/22/9/1790/T2.expansion.html |
| transFac_score | regulation | TransFac v7.0 score, 0-1000 |
| transFac_name | regulation | TransFac v7.0 name |
| miRNA | regulation | miRbase miRNA |
| targetScan | regulation | targetScan |
| segDup | repeat | segmental duplication scores, duplications of >1 kb of non- |
| | | repeat-masked sequence with >90% sequence similarity, 0-1 |
| rmsk_name | repeat | Repetitive element name from RepeatMasker |
| rmsk_class | repeat | Repetitive element class from RepeatMasker |
| rmsk_family | repeat | Repetitive element family from RepeatMasker |
| hapmap2and3_ASW | allele_frequency | ASW hapmap population allele frequency |
| hapmap2and3_CEU | allele_frequency | CEU hapmap population allele frequency |
| hapmap2and3_CHB | allele_frequency | CHB hapmap population allele frequency |
| hapmap2and3_CHD | allele_frequency | CHD hapmap population allele frequency |
| hapmap2and3_GIH | allele_frequency | GIH hapmap population allele frequency |
| hapmap2and3_JPT | allele_frequency | JPT hapmap population allele frequency |
| hapmap2and3_LWK | allele_frequency | LWK hapmap population allele frequency |
| hapmap2and3_MEX | allele_frequency | MEX hapmap population allele frequency |
| hapmap2and3_MKK | allele_frequency | MKK hapmap population allele frequency |
| hapmap2and3_TSI | allele_frequency | TSI hapmap population allele frequency |
| hapmap2and3_YRI | allele_frequency | YRI hapmap population allele frequency |
| 1000g2010nov_ALL | allele_frequency | 1000 genomes November 2010 release, pilot 1, all subjects |
| 1000g2011may_ALL | allele_frequency | 1000 genomes May 2011 release, phase 1, all subjects |
| 1000g2012feb_ALL | allele_frequency | 1000 genomes February 2012 release, phase 1, all subjects |
| 1000g2012apr_ALL | allele_frequency | 1000 genomes April 2012 release, phase 1, all subjects |
| 1000g2012apr_EUR | allele_frequency | 1000 genomes April 2012 release, phase 1, European ancestry |
| 1000g2012apr_AFR | allele_frequency | 1000 genomes April 2012 release, phase 1, West African ancestry |
| 1000g2012apr_AMR | allele_frequency | 1000 genomes April 2012 release, phase 1, American ancestry |
| 1000g2012apr_ASN | allele_frequency | 1000 genomes April 2012 release, phase 1, East Asian ancestry |
| cg46 | allele_frequency | Complete genomics diversity panel (46 unrelated subjects) |
| cg69 | allele_frequency | Complete genomics public panel (69 subjects, including 17 |
| | | member CEPH pedigree) |
| dbSNP137 | allele_frequency | DBsnp 137 |
| esp6500si_ALL | allele_frequency | ESP 6500 subjects, all, including indels |
| esp6500si_EA | allele_frequency | ESP 6500 subjects, european american, including indels |
| esp6500si_AA | allele_frequency | ESP 6500 subjects, african american, including indels |
| esp_pi | allele_frequency | Nucleotide diversity From ESP 2440, as quantified by pi, mean |
| | | number of pairwise differences between two randomly selected |
| | | chromosomes for the gene region, scaled to gene length |
| | | (Science 2012: 337; 64-69) |
| dbnsfp_PhyloP | functional_prediction | placental subset of site-wise conservation score PhyloP score |
| | | from dbNSFP, 0-1 |
| dbnsfp_PhyloP_Pred | functional_prediction | placental subset of site-wise conservation score |
| | | PhyloPprediction from dbNSFP, C = conserved, N = neutral |
| dbnsfp_SIFT | functional_prediction | SIFT score from dbNSFP, 0-1 |
| dbnsfp_SIFT_Pred | functional_prediction | SIFT prediction, D = damaging, T = tolerated |
| dbnsfp_PolyPhen2 | functional_prediction | HumDiv trained PolyPhen scores from dbNSFP, 0-1 |
| dbnsfp_PolyPhen2_Pred | functional_prediction | HumDiv trained PolyPhen prediction from dbNSFP, D = probably |
| | | damaging, P = possibly damaging, B = benign |
| dbnsfp_LRT | functional_prediction | LRT test of codon constraint from dbNSFP, 0-1 |
| dbnsfp_LRT_Pred | functional_prediction | LRT prediction of effect of NS variant from dbNSFP, |
| | | D = deleterious, N = neutral |
| dbnsfp_MutationTaster | functional_prediction | Mutation Taster score for ensembl NS SNVs from dbNSFP 0-1 |
| dbnsfp_MutationTaster_Pred | functional_prediction | Mutation Taster score for ensembl NS SNVs from dbNSFP, |
| | | A = disease causing automatic, D = disease causing, N = |
| | | polymoprhism, P = polymorphism automatic |
| dbnsfp_GERP++ | functional_prediction | GERP++ predictions for NS SNVs from dbNSFP |
| wg_GERP++_gt2 | functional_prediction | whole genome gerp scores, including synonymous variants |
| | | and noncoding regions if >2 |
| phastCons_46wayMSA_score | functional_prediction | phastCons most conserved element score, 0-1000 |
| phastCons_46wayMSA_lod | functional_prediction | phastCons most conserved element lod score |
| gwasCatalog_disease | phenotype_association | phenotype association as reported in NHGRI GWAS catalog |
| gwasCatatog_risksnp | phenotype_association | risk variant as reported in NHGRi GWAS catalog |
| gwasCatalog_OR | phenotype_association | odds ratio as reported in NHGRI GWAS catalog |
| gwasCatalog_pval | phenotype_association | p value as reported in NHGRI GWAS catalog |
| clinvar_variant_disease | phenotype_association | variant disease annotation according to clinvar vcf in dbvar |
| clinvar_variant_classification | phenotype_association | variant classification according to clinvar vcf in dbvar |
| clinvar_variant_type | phenotype_association | variant type according to clinvar vcf in dbvar |
| clinvar_variant_source | phenotype_association | variant data source according to clinvar vcf in dbvar |
| clinvar_variant_sourceID | phenotype_association | variant data source ID according to clinvar vcf in dbvar |
| hgmd_url | phenotype_association | url for public HGMD entry for variant |
| clinvar_gene_disease | phenotype_association | Clinvar disease associated with gene (NOT variant) |
| omim_gene_disease | phenotype_association | OMIM disease associated with gene (NOT variant) |
| omim_url | phenotype_association | url for OMIM entry |
| cosmic_id | phenotype_association | COSMIC database id for somatic cancer mutations |
| cosmic_occurrence | phenotype_association | tissue occurrence in COSMIC |
| geneReviews | phenotype_association | url for GTR Gene Reviews description of gene locus |
| pharmGKB_clin | phenotype_association | pharmGKB basic clnical annotation (drug, class of association, |
| | | evidence level). Description of evidence |
| | | level: http://www.pharrngkb.org/page/clinAnnLevels |
| pharmGKB_url | phenotype_association | pharmGKB url for clinical snp annotation |
| CHROM | vcf_info | vcf chromosome |
| POS | vcf_info | vcf position |
| ID | vcf_info | vcf rsid |
| REF | vcf_info | vcf reference base |
| ALT | vcf_info | vcf alt base |
| QUAL | vcf_info | vcf quality score |
| FILTER | vcf_info | vcf filter field |
| INFO | vcf_info | vcf info field |
| FORMAT | vcf_info | vcf format field |
| CALLS | vcf_info | vcf calls |

| | | Delimiters |
|---|---|---|
| | | for multiple |
| Field | Comment | matches |
| function | priority for function assignment: | ; |
| | http://www.openbioinformatics.org/annovar/annovar_gene.html#output1 | |
| gene_knowngene | | , for multiple |
| | | gene matches, |
| | | ; for multiple |
| | | variant function |
| | | definitions for |
| | | the same gene |
| | | as described in |
| | | function field |
| exonicFunction_knowngene | priority for exonic function assignment: | |
| | http://www.openbioinformatics.org/annovar/annovar_gene.html#output2 | |
| aaChange_knowngene | | , |
| exonicFunction_gencode | priority for exonic function assignment: | |
| | http://www.openbioinformatics.org/annovar/annovar_gene.html#output2 | |
| aaChange_gencode | | , |
| exonicFunction_refgene | priority for exonic function assignment: | |
| | http://www.openbioinformatics.org/annovar/annovar_gene.html#output2 | |
| aaChange_refgene | | , |
| pfamA_domain | | \| |
| bioCyc_id | | \| |
| bioCyc_pathway | | \| |
| kegg_id | | \| |
| kegg_pathway | | \| |
| pseudogene_id | | \| |
| pseudogene_class | | \| |
| has_pseudogene_eValue | lower value indicates higher degree of sequence homology | \| |
| has_pseudogene_name | | \| |
| encode_dnaseCluster | | |
| encode_tfbsChip_score | | |
| encode_tfbsChip_name | | |
| encode_HMMgm12878 | | |
| encode_HMMhesc | | |
| encode_HMMhsmm | | |
| encode_HMMhuvec | | |
| encode_HMMnhek | | |
| encode_HMMnhlf | | |
| cpg_Island | | |
| regulome_cat1_annotation | | |
| regulome_cat1_ class | | |
| transFac_score | | \| |
| transFac_name | | \| |
| miRNA | | |
| targetScan | | |
| segDup | only scores >0.9 are output | |
| rmsk_name | | \| |
| rmsk_class | | \| |
| rmsk_family | | \| |
| hapmap2and3_ASW | lifted over from hg18 | |
| hapmap2and3_CEU | lifted over from hg18 | |
| hapmap2and3_CHB | lifted over from hg18 | |
| hapmap2and3_CHD | lifted over from hg18 | |
| hapmap2and3_GIH | lifted over from hg18 | |
| hapmap2and3_JPT | lifted over from hg18 | |
| hapmap2and3_LWK | lifted over from hg18 | |
| hapmap2and3_MEX | lifted over from hg18 | |
| hapmap2and3_MKK | lifted over from hg18 | |
| hapmap2and3_TSI | lifted over from hg18 | |
| hapmap2and3_YRI | lifted over from hg18 | |
| 1000g2010nov_ALL | | |
| 1000g2011may_ALL | | |
| 1000g2012feb_ALL | | |
| 1000g2012apr_ALL | | |
| 1000g2012apr_EUR | | |
| 1000g2012apr_AFR | | |
| 1000g2012apr_AMR | | |
| 1000g2012apr_ASN | | |
| cg46 | | |
| cg69 | | |
| dbSNP137 | | |
| esp6500si_ALL | | |
| esp6500si_EA | | |
| esp6500si_AA | | |
| esp_pi | | |
| dbnsfp_PhyloP | on dbNSFP scale (larger score is more conserved) | |
| dbnsfp_PhyloP_Pred | http://onlinelibrary.wiley.com/doi/10.1002/humu.21517/full | |
| dbnsfp_SIFT | on dbNSFP scale (larger score is more deleterious) | |
| dbnsfp_SIFT_Pred | http://onlinelibrary.wiley.com/doi/10.1002/humu.21517/full | |
| dbnsfp_PolyPhen2 | on dbNSFP scale (larger score is more deleterious) | |
| dbnsfp_PolyPhen2_Pred | http://onlinelibrary.wiley.com/doi/10.1002/humu.21517/full | |
| dbnsfp_LRT | on dbNSFP scale (larger score is more constrained) | |
| dbnsfp_LRT_Pred | http://onlinelibrary.wiley.com/doi/10.1002/humu.21517/full | |
| dbnsfp_MutationTaster | on dbNSFP scale (larger score is more deleterious) | |
| dbnsfp_MutationTaster_Pred | | |
| dbnsfp_GERP++ | http://onlinelibrary.wiley.com/doi/10.1002/humu.21517/full | |
| wg_GERP++_gt2 | only scores >2 are output | |
| phastCons_46wayMSA_{——}score | DOES NOT match alt allele from vcf file to risk variant | \| |
| phastCons_46wayMSA_lod | DOES NOT match alt allele from vcf file to risk variant | \| |
| gwasCatalog_disease | DOES NOT match alt allele from vcf file to risk variant | \| |
| gwasCatatog_risksnp | DOES NOT match alt allele from vcf file to risk variant | \| |
| gwasCatalog_OR | DOES NOT match alt allele from vcf file to risk variant | \| |
| gwasCatalog_pval | DOES NOT match alt allele from vcf file to risk variant | \| |
| clinvar_variant_disease | | |
| clinvar_variant_classification | | |
| clinvar_variant_type | | |
| clinvar_variant_source | | |
| clinvar_variant_sourceID | | |
| hgmd_url | | \| |
| clinvar_gene_disease | Matches on gene region, NOT variant | \| |
| omim_gene_disease | Matches on gene region, NOT variant; does NOT report | \| |
| | susceptibility ({ } in OMIM) or non-disease phenotype loci ([ ] | |
| | in OMIM): http://www.omim.org/help/faq#1.6 | |
| omim_url | Matches on gene region, NOT variant; does NOT report | \| |
| | susceptibility ({ } in OMIM) or non-disease phenotype loci ([ ] | |
| | in OMIM): http://www.omim.org/help/faq#1.6 | |
| cosmic_id | | \| |
| cosmic_occurrence | | \| |
| geneReviews | Matches on gene region, NOT variant | \| |
| pharmGKB_clin | DOES NOT match genotype to drug annotation | \| |
| pharmGKB_url | DOES NOT match genotype to drug annotation | |
| CHROM | | |
| POS | | |
| ID | | |
| REF | | |
| ALT | | |
| QUAL | | |
| FILTER | | |
| INFO | | |
| FORMAT | | |
| CALLS | | |

### Appendix B

TABLE 2

| Field | Category | Description |
|---|---|---|
| function | gene | variant function according to UCSC knowngene |
| gene_knowngene | gene | gene according to UCSC knowngene |
| pfamA_domain | gene | pfam level A annotations (manually curated) for protein family/domain: |
| | | http://pfam.sanger.ac.uk/ |
| bioCyc_Id | gene | bioCyc identifier http://biocyc.org |
| bioCyc_pathway | gene | bioCyc pathway http://biocyc.org |
| kegg_Id | gene | KEGG id |
| kegg_pathway | gene | KEGG pathway |
| pseudogene_Id | gene | is variant in a pseudogene according to Yale Pseudogene database 70, pseudogene |
| | | id |
| pseudogene_class | gene | is variant in a pseudogene according to Yale Pseudogene database 70, pseudogene |
| | | class |
| has_pseudogene_eValue | gene | is variant in a gene with sequence similarity to a known pseudogene, according to |
| | | ensembl/Yale intersection, blast e value, 0-1 |
| has_pseudogene_name | gene | is variant in a gene with sequence similarity to a known pseudogene, according to |
| | | ensembl/Yale intersection, pseudogene name |
| encode_dnaseCluster | regulation | encode DNAse hypersensitivity cluster score, 0-1000 |
| encode_tfbsChip_score | regulation | encode TFBS CHIP score, 0-1000 |
| encode_tfbsChip_name | regulation | encode TFBS CHIP factor name |
| encode_HMMgm12878 | regulation | HMM-based classification of regulatory regions in lymphoblastoid cells from |
| | | NA12878 |
| encode_HMMhesc | regulation | HMM-based classification of regulatory regions in IH1-hESC |
| encode_HMMhsmm | regulation | HMM-based classification of regulatory regions in skeletal muscle myoblasts |
| encode_HMMhuvec | regulation | HMM-based classification of regulatory regions in umbilical vein endothelial cells |
| encode_HMMnhek | regulation | HMM-based classification of regulatory regions in epidermal keratinocytes |
| encode_HMMnhlf | regulation | HMM-based classification of regulatory regions in lung fibroblasts |
| cpg_Island | regulation | CpG Islands, cpg number |
| transFac_score | regulation | TransFac v7.0 score, 0-1000 |
| transFac_name | regulation | TransFac v7.0 name |
| miRNA | regulation | miRbase miRNA |
| targetScan | regulation | targetScan |
| segDup | repeat | segmental duplication scores, duplications of >1 kb of non-repeat-masked sequence |
| | | with >90% sequence similarity, 0-1 |
| rmsk_name | repeat | Repetitive element name from RepeatMasker |
| rmsk_class | repeat | Repetitive element class from RepeatMasker |
| rmsk_family | repeat | Repetitive element family from RepeatMasker |
| dgv_occurrences | allele_frequency | number of occurrrences of any SV in dgv overlapping called SV by one or more |
| | | base pairs |
| decipher_occurrences | allele_frequency | number of occurrrences of any SV in decipher overlapping called SV by one or |
| | | more base pairs |
| decipher_type | allele_frequency | type of SV cataloged in decipher |
| decipher_phenotype | allele_frequency | phenotype associated with SV in decipher |
| NIMH_event | allele_frequency | event type observed in NIMH controls |
| Campbell_discrete_frequency | allele_frequency | Genotype frequencies for discrete cnps cataloged by Campbell, et al. Am J Hum |
| | | Genet. 2011 Mar. 11; 88(3): 317-32. |
| Campbell_nondiscrete_copyn | allele_frequency | Median copy number for cnps cataloged by Campbell, et al. Am J Hum Genet. 2011 |
| | | Mar. 11; 88(3): 317-32. |
| clinvar_gene_disease | phenotype_association | Clinvar disease associated with gene (NOT variant) |
| omim_gene_disease | phenotype_association | OMIM disease associated with gene (NOT variant) |
| omim_url | phenotype_association | url for OMIM entry |
| cosmic_Id | phenotype_association | COSMIC database id for somatic cancer mutations |
| cosmic_occurrence | phenotype_association | tissue occurrence in COSMIC |
| geneReviews | phenotype_association | url for GTR Gene Reviews description of gene locus |
| chrom | phenotype_association | gff chromosome |
| start | phenotype_association | gff start |
| end | gff_info | gff end |
| type | gff_info | gff type of SV |
| filter | gff_info | gff filter |
| events | gff_info | gff # of events |

| | | Delimiters |
|---|---|---|
| | | for multiple |
| Field | Comment | matches |
| function | priority for function assignment: | ; |
| | http://www.openbioinformatics.org/annovar/annovar_gene.html#output1 | |
| gene_knowngene | | , for multiple |
| | | gene matches, |
| | | ; for multiple |
| | | variant function |
| | | definitions for |
| | | the same gene |
| | | as described in |
| | | function field |
| pfamA_domain | | \| |
| bioCyc_Id | | \| |
| bioCyc_pathway | | \| |
| kegg_Id | | \| |
| kegg_pathway | | \| |
| pseudogene_Id | | \| |
| pseudogene_class | | \| |
| has_pseudogene_eValue | lower value indicates higher degree of sequence | \| |
| | homology | |
| has_pseudogene_name | | \| |
| encode_dnaseCluster | | |
| encode_tfbsChip_score | | |
| encode_tfbsChip_name | | |
| encode_HMMgm12878 | | |
| encode_HMMhesc | | |
| encode_HMMhsmm | | |
| encode_HMMhuvec | | |
| encode_HMMnhek | | |
| encode_HMMnhlf | | |
| cpg_Island | | |
| transFac_score | | \| |
| transFac_name | | \| |
| miRNA | | \| |
| targetScan | | |
| segDup | only scores >0.9 are output | |
| rmsk_name | | \| |
| rmsk_class | | \| |
| rmsk_family | | \| |
| dgv_occurrences | | |
| decipher_occurrences | | |
| decipher_type | | |
| decipher_phenotype | | |
| NIMH_event | | |
| Campbell_discrete_frequency | | |
| Campbell_nondiscrete_copyn | | |
| clinvar_gene_disease | Matches on gene region, NOT variant | \| |
| omim_gene_disease | Matches on gene region, NOT variant; does NOT report | \| |
| | susceptibility ({ } in OMIM) or non-disease phenotype | |
| | loci ([ ] in OMIM): http://www.omim.org/help/faq#1.6 | |
| omim_url | Matches on gene region, NOT variant; does NOT report | \| |
| | susceptibility ({ } in OMIM) or non-disease phenotype | |
| | loci ([ ] in OMIM): http://www.omim.org/help/faq#1.6 | |
| cosmic_Id | | \| |
| cosmic_occurrence | | \| |
| geneReviews | Matches on gene region, NOT variant | \| |
| chrom | | \| |
| start | | |
| end | | |
| type | | |
| filter | | |
| events | | |

What is claimed is:

1. A computerized method for identifying clinical phenotypes, comprising:
receiving data corresponding to sequence alignment and variant identification for a plurality of single nucleotide variants or indels;
performing a pipelined process among a set of processing units, wherein the pipelined process comprises
comparing the received variant identification information with a dataset of rare SNVs, SVs, or indels, wherein the variant identification comparison is performed among a first subset of the set of processing units,
comparing the sequence alignment information with a dataset of rare disease risk genotypes, wherein the sequence alignment comparison is performed among a second subset of the set of processing units,
comparing the sequence alignment information with a dataset of drug response genotypes, wherein the sequence alignment comparison is performed among a third subset of the set of processing units;
identifying a set of rare variants in inherited disease genes based on the variant identification comparison and the sequence alignment comparison, wherein the identified set of rare variants meets a first set of predetermined criteria;
identifying a set of known variants in inherited disease genes based on the variant identification comparison and the sequence alignment comparison, wherein identified set of known variants meets a second set of predetermined criteria;
identifying a set of pharmacogenomics haplotypes based on the sequence alignment comparison, wherein the identified set of pharmacogenomics haplotypes meets a third set of predetermined criteria;
identifying a set of pharmacogenomics genotypes based on the sequence alignment comparison, wherein the identified set of pharmacogenomics genotypes meets a fourth set of predetermined criteria.

2. The method of claim 1, further comprising categorizing the identified set of rare variants into a first plurality of tiers.

3. The method of claim 1, further comprising categorizing the identified set of known variants into a second plurality of tiers.

4. The method of claim 1, further comprising categorizing the identified set of pharmacogenomics haplotypes into a third plurality of tiers.

5. The method of claim 1, further comprising categorizing the identified set of pharmacogenomics genotypes into a fourth plurality of tiers.

6. The method of claim 1, wherein identifying of the set of rare variants in inherited disease genes is performed among a fourth subset of the set of processing units.

7. The method of claim 1, wherein identifying a set of known variants in inherited disease genes is performed among a fifth subset of the set of processing units.

8. The method of claim 1, wherein identifying a set of pharmacogenomics haplotypes is performed among a sixth subset of the set of processing units.

9. The method of claim 1, wherein identifying a set of pharmacogenomics genotypes is performed among a seventh subset of the set of processing units.

10. The method of claim 1, wherein the set of rare variants in inherited disease genes and the set of known variants in inherited disease genes are collected as a set of inherited disease risk candidates.

11. The method of claim 1, wherein the set of pharmacogenomics haplotypes and the set of pharmacogenomics genotypes are collected as a set of drug response candidates.

12. A non-transitory computer-readable medium including instructions that, when executed by a processing unit, cause the processing unit to identify clinical phenotypes, by performing the steps of:
receiving data corresponding to sequence alignment and variant identification for a plurality of single nucleotide variants or indels;
performing a pipelined process among a set of processing units, wherein the pipelined process comprises
comparing the received variant identification information with a dataset of rare SNVs, SVs, or indels, wherein the variant identification comparison is performed among a first subset of the set of processing units,
comparing the sequence alignment information with a dataset of rare disease risk genotypes, wherein the sequence alignment comparison is performed among a second subset of the set of processing units,
comparing the sequence alignment information with a dataset of drug response genotypes, wherein the sequence alignment comparison is performed among a third subset of the set of processing units;
identifying a set of rare variants in inherited disease genes based on the variant identification comparison and the sequence alignment comparison, wherein the identified set of rare variants meets a first set of predetermined criteria;
identifying a set of known variants in inherited disease genes based on the variant identification comparison and the sequence alignment comparison, wherein identified set of known variants meets a second set of predetermined criteria;
identifying a set of pharmacogenomics haplotypes based on the sequence alignment comparison, wherein the identified set of pharmacogenomics haplotypes meets a third set of predetermined criteria;
identifying a set of pharmacogenomics genotypes based on the sequence alignment comparison, wherein the identified set of pharmacogenomics genotypes meets a fourth set of predetermined criteria.

13. The non-transitory computer-readable medium of claim 12, further comprising categorizing the identified set of rare variants into a first plurality of tiers.

14. The non-transitory computer-readable medium of claim 12, further comprising categorizing the identified set of known variants into a second plurality of tiers.

15. The non-transitory computer-readable medium of claim 12, further comprising categorizing the identified set of pharmacogenomics haplotypes into a third plurality of tiers.

16. The non-transitory computer-readable medium of claim 12, further comprising categorizing the identified set of pharmacogenomics genotypes into a fourth plurality of tiers.

17. The non-transitory computer-readable medium of claim 12, wherein identifying of the set of rare variants in inherited disease genes is performed among a fourth subset of the set of processing units.

18. The non-transitory computer-readable medium of claim 12, wherein identifying a set of known variants in inherited disease genes is performed among a fifth subset of the set of processing units.

19. The non-transitory computer-readable medium of claim 12, wherein identifying a set of pharmacogenomics haplotypes is performed among a sixth subset of the set of processing units.

20. The non-transitory computer-readable medium of claim 12, wherein identifying a set of pharmacogenomics genotypes is performed among a seventh subset of the set of processing units.
